(19) United States
(12) Patent Application Publication
(10) Pub. No.: US 2011/0253137 A1
(43) Pub. Date: Oct. 20, 2011

(54) System to control a patient's ventilator or anesthesia machine

(76) Inventors: Anthony Calderoni, Bristol, CT (US); Rogers Pylant, Kensington, CT (US); Michael Calderoni, Farmington, CT (US); Ardeshir Dehkordi, New Haven, CT (US)

(21) Appl. No.: 13/066,249

(22) Filed: Apr. 8, 2011

Related U.S. Application Data
(60) Provisional application No. 61/342,045, filed on Apr. 8, 2010.

(51) Int. Cl. A61M 16/04; A61M 16/01; A61M 16/00

(52) U.S. Cl. 128/203.12; 128/207.15; 128/205.13

(57) ABSTRACT

A system for the control of a ventilator or an anesthesia delivery machine for an intubated patient, wherein the intubated patient has an endotracheal tube inserted into the airway passage, the endotracheal tube having a controllably inflatable annular cuff thereon, wherein the cuff is monitored and controlled by a pressure control unit, the system including: a patient-disturbed signal arrangement for providing a feedback to the ventilator or anesthesia delivery machine through the pressure control unit to delay pressure changes directed to the cuff by the ancillary devices.

### BACKGROUND OF THE INVENTION

1. Field of the Invention

This invention relates to intubation devices and more particularly to control systems for monitoring patients during their intubation process and providing control of that intubation process, and is based upon Provisional Patent Application No. 61/342,045, filed Apr. 8, 2010, and co-filed with commonly owned application ARM-12 NonProv, Ser. No. 12/______, filed 8 Apr. 2011, each of which are incorporated herein by reference in their entirety.

2. Prior Art Discussion

Human medical patients are supplied with artificial ventilation during surgery or in emergency situations. These procedures require intubation of the patient using an endotracheal tube or a tracheostomy tube. Intubation of a patient is a procedure which is useful, yet potentially harmful if not controlled.

Intubation is accomplished by insertion of an endotracheal tube, ETT (or tracheostomy tube, TT) through the patient's mouth (or tracheostomy) into the airway passage or trachea. These devices generally comprise a relatively pliable tool with a means for connecting it to a respirator, ventilator, anesthesia machine, or other air supply mechanism for the introduction of air into the lungs. ETT and TT tubes include an inflatable/deflatable bag-like structure or balloon “cuff” around the exterior of the distal end of the ETT or TT tube. The balloon cuff is typically located in a position along the ETT or TT tube so as to engage the inner wall of the trachea.

A major concern for the placement of an ETT or TT tube cuff is the cuffs pressure management. Typically this has been accomplished by the manual palpation of the pilot balloon found on the cuff lumen on most major ETT or TT tube manufacturer's products. Alternatively, a manometer is utilized to measure the endotracheal tube cuff pressure directly. However, a manometer will not regulate the cuff pressure.

Cuff pressure should be about 45 cm of water as the maximum desirable amount. Over-inflation may cause tracheal necrosis, stenosis, and rupture of the patient's passageway in which the cuff is inserted. Under-inflation of the ETT or TT tube cuff to a pressure below about 20 to 25 cm of water may allow silent aspiration of pharyngeal secretions thought to contribute to the contraction of ventilator associated pneumonia (VAP). Air leakage from the ETT or TT tube cuff may cause a cuff pressure drop to below levels considered adequate to prevent aspiration of fluids. In anesthesia, nitrous oxide gases permeate the ETT or TT tube cuff walls causing undesirable and possible dangerous increases in cuff pressure. Moreover, less than a fully formed cuff allows pathways for contaminated sub glottic secretions to migrate past the cuff and into the lungs of the patient.

It is an object of the present invention to overcome the disadvantages of the prior art.

It is a further object of the present invention to provide a system for cuff management of an associated ETT or TT tube maintained within a patient's airway.

It is a further object of the present invention to provide a cuff monitoring and control arrangement for an ETT or TT tube, wherein undesired over-pressure and under-pressure is avoided by the cuff pressure maintenance system.

It is a further object of the present invention to provide safeguards that perform so as to limit any injury to the patient should circuits or software fail. In one case, a self test of the cuff management control loop is scheduled every 15 minutes to affirm the proper operation of the control mechanisms, Secondly, a fail-safe cuff pressure reducing mechanism has been devised should the cuff pressure rise to dangerous levels due to failure of the control mechanism, or environmental forces.

It is yet a further object of the present invention to provide a system for the safe control of ancillary devices which supply treatment options to a patient undergoing intubation therapy.

It is another object of the present invention to provide a system for controlling machines utilized with endotracheal tubes, those machines being ventilators, anesthesia machines or resuscitation devices and to prevent dangerous operating parameters therewith.

### BRIEF SUMMARY OF THE INVENTION

The present invention comprises a cuff pressure regulator and control system, which system includes and is arranged to monitor and control an endotracheal (ETT or TT) tube in a patient. The regulator and control system fluidly communicates with and inflates and adjusts the pressure within the ETT or TT tube cuff at the distal end of the ETT or TT tube, once the ETT or TT tube has been inserted into the patient's trachea by a respiratory technician. The technician sets a desired pressure on the regulatory and control system typically within a plus or minus 2 cm of water pressure. The regulator and control system controls the pressure in the cuff by fluid connective means communicating with a small air pump, a pressure sensor and a dump valve. A pressure feedback loop with a microprocessor controller contained within the regulator and control system has direct control over both of the air pump and the dump valve.

The pressure regulator and control system monitors the fluid pressure in the cuff and adjusts the cuff pressure automatically to the pressure preselected by the technician. The pneumatic circuit includes the tube having a cuff disposed annularly at one end thereof. A conduit extends from the cuff through a line in communication with a pressure relief switch, and with further communication downstream with a dump valve and further downstream with a check valve, all in controlled, responsive fluid communication with the air pump.

The pressure regulator and control system may be also described in a block diagram manner controlled by a central microprocessor therein. The microprocessor has a liquid crystal display panel with associated proper control switches. The microprocessor is programmed and is supported through a program port, a time and date clock mechanism and a battery gas gauge. The battery gas gauge is supported by a power system which includes a 6 V wall cube power supply, Li-ion batter pack, and a power manager charger.

The microprocessor is in electrical communication and supports an alarm light emitting diode and an audible alarm buzzer. The microprocessor controls the air pump. The air pump is in fluid communication with a pneumatic circuit. The pneumatic circuit is in communication with the pressure sensor which reports back to the microprocessor through a proper circuit. The microprocessor also controls the use of the dump valve. The safety pressure relief switch monitors the pneumatic circuit pressure (and therefore the cuff pressure) for violation of the high limit cuff pressure trigger point. Once the cuff pressure reaches the high limit cuff pressure trigger point, the safety pressure relief switch energizes the dump valve resulting in a lowering of the cuff pressure to safe levels without the microprocessor control. The lowering of the cuff pressure below the trigger point of the safety pressure relief switch results from inherent hysteresis and time delay of the mechanical switch remaining open long after the cuff pressure is reduced below the initial trigger point. The pneumatic circuit will thereby be kept open to atmospheric pressure preventing dangerous cuff pressures (above 45 cm H2O) to persist for more than a few seconds. The pneumatic circuit is in fluid communication with an output port which is in fluid communication with the tube cuff disposed within the patient.

The microprocessor of the regulator and control system measures the fluid pressure in the tube cuff and automatically adjusts the air pressure inside the tube cuff to the level selected by the technician. That pressure level is constantly sensed by the pressure sensor during the term of use of that cuff within the patient's trachea. The automatic pressure in the cuff is regulated by selective microprocessor activation of either the air pump or by microprocessor activation of the dump valve to lower the pressure in the tube cuff, all as sensed, as necessary, by the microprocessor and its attached reporting units. The pressure relief of an overinflated cuff may in one embodiment, be delayed for a very limited time to insure that any increase in pressure is only temporary, and need not be acted upon. Unnecessary pressure relief could be dangerous in that aspiration of sub-glottic fluids may occur if such pressure relief were too great, and/or unnecessary. Tugging on the conduit, or coughing, moving or other action by the patient will cause such temporary increase in the pressure in the cuff, which would otherwise be temporary. The safety pressure relief switch however, will act as a safety override to ensure that the pressure in the tube cuff does not exceed 45 cm of water for extended time pressure.

The regulation and control system of the present invention provides a constant feedback of the patient's position and cuff pressure at all times during its use. A bed head-end angle sensor is preferably attached to the bed of the patient to ensure that the head of the bed is inclined at an angle of at least 30°. The sensor is in communication with the microprocessor through a proper circuit. If the angle of the head end of the patient's bed is not at an angle of at least about 30 degrees, the microprocessor receives the angle alert signal and then triggers multiple alarms to show and sound such improper head of bed angle position or condition. An audible and a visible alarm are also automatically timed at periodic intervals by the microprocessor, as part of the intubation monitoring and control system, to provide a reminder to the technician to maintain the care of the patient's mouth, for swabbing while section with an antibacterial solution and/or to remove sub glottic or oral secretions from within the mouth.

A further embodiment of the intubation monitoring and control system comprises a temporary initial controlled pressure surge by the air pump, of pressure in the tube cuff from its preferred range of to 40 cm of water, to a very brief, higher pressure of about 65 cm of water, so as to remove any wrinkles or creases within the tube cuff, thereby avoiding leakages past the cuff in the trachea.

The invention thus comprises an endotracheal tube or tracheostomy tube monitoring and control system for the safe and effective use of a pressurizable annular cuff about the distal end of that tube arranged within the larynx or trachea of a human patient comprising: a control unit for the monitoring and control of the tube cuff in communication therewith; a pressure sensor in communication with the control unit and with an empowered air pump and associated a dump valve; and a feedback circuit connected to an ancillary device supporting the patient, so as to control time and pressure responsive use of the ancillary device relative to pressure changes in the tracheal cuff in the patient. The ancillary devices are selected from the group consisting of: a ventilator, an anesthesia machine and a resuscitation bag. The pressure in the cuff is pre-set and maintained at a selected value by an system operator, and wherein the system provides feedback of pressure within the cuff and acts to dump pressure from that cuff after a predetermined delay to avoid unnecessary and potentially damaging pressure increase. The system includes a self-test control unit protocol which automatically checks operation of the pneumatic system control components to assure that the microprocessor continues to have control over system integrity for feedback to the ancillary device. The system further includes a pressure sensor in fluid communication with a dump valve and an air pump, controlled by a control unit arranged to delay pressure changes in the ancillary device connected to the system and to delay pressure changes effected by the system on the cuff. The system also includes a programming port for inputting to the control unit, instructions for the pressure and frequency overrides on the system's ancillary device, a dual alarm arrangement for establishing a periodic review of a patient utilizing the system, a pneumatic circuit controlled by the control unit, the circuit consisting of a pressure sensor, an air pump, a dump valve, a safety pressure relief switch attached to the dump valve, and an output port, all in controlled fluid communication with the cuff. The output port communicates directly with the cuff, the output port being pressurized directly by the air pump which is controlled by the pressure sensor and the control unit. A safety pressure relief switch to override the microprocessor control should cuff pressures reach dangerous levels above 45 cm H2O.

The invention also includes a method to control ancillary devices supporting a patient having an endotracheal tube inserted within the patient's trachea, the method comprising: a system controlled by a control unit, which unit controls timed pressure intervention in a cuff on the tracheal tube; a delay arrangement within the control unit to delay an decrease in pressure in the cuff in response to a signal received by the control unit, so as to avoid pressure changes responsive to temporary increases in cuff pressure. The delay of a decrease in pressure in the cuff in response to a received signal by the control unit of an improper increase in pressure in the cuff is about 2 seconds. The delay of a decrease in pressure in the cuff in response to a received signal by the control unit of an improper increase in pressure in the cuff may also be about 5 seconds to about 10 seconds.

The invention also includes a method for monitoring and controlling use of an endotracheal or tracheal tube cuff in a patient's trachea of a patient lying in a bed, comprising: attaching a pressurized line to a tube cuff for engaging the patient's trachea; monitoring and controlling the pressure in the cuff in the patient by a monitor and control system; limiting the pressure in the cuff to about 45 inches of water; sounding a patient monitoring alarm by the system, to remind a patient assist personnel to check and cleanse the patient's airway; sounding a patient monitoring alarm by the system, if the angle of the bed is less than about 30 degrees from horizontal; overriding control from the microprocessor by the safety pressure relief switch in response to a received danger signal, should cuff pressures rise to dangerous levels; and controlling ancillary devices from introducing pressure increases into the cuff. The method includes communicating with an ancillary patient device to provide an immediate indication of patient discomfort, wherein the ancillary device is a ventilator or wherein the ancillary device is a anesthesia device. The method may include lowering the cuff pressure after a timed delay after the received danger signal has been picked up by the microprocessor, wherein the time delay is about 2 seconds before pressure is reduced, or wherein the time delay is about 5 to 10 seconds before pressure in the cuff is reduced.

### BRIEF DESCRIPTION OF THE DRAWINGS

The objects and advantages of the present invention will become more apparent when viewed in conjunction with the following drawings in which:

FIG. 1 is a pneumatic circuit diagram of a cuff pressure regulator and control system constructed according to the principles of the present invention;

FIG. 2 is a side elevation partial view of a human patient showing an endotracheal tube with an inflatable cuff adjacent its distal end, within that patient's airway, along with an angle sensed and controlled bed for the patient;

FIG. 3 is a block diagram of the cuff pressure regulator and control system arranged according to the preferred principles of the present invention;

FIG. 4 is a flow chart representing the steps involved in controlling the pressure in a cuff within an intubated patient; and

FIG. 5 is a flow chart representing the steps the system utilizes in its running operation.

### DETAILED DESCRIPTION OF THE DRAWINGS

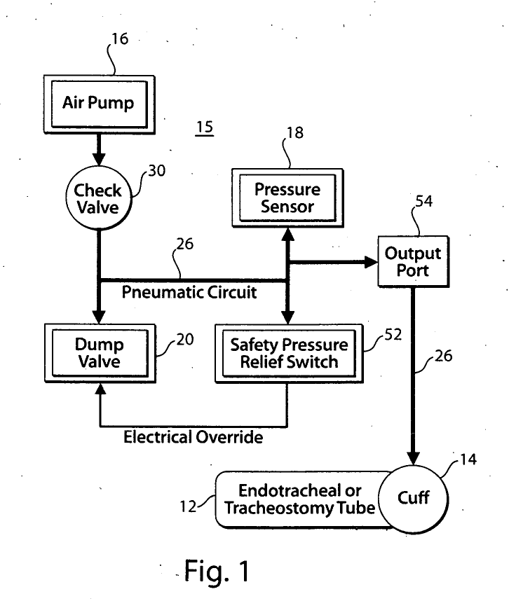

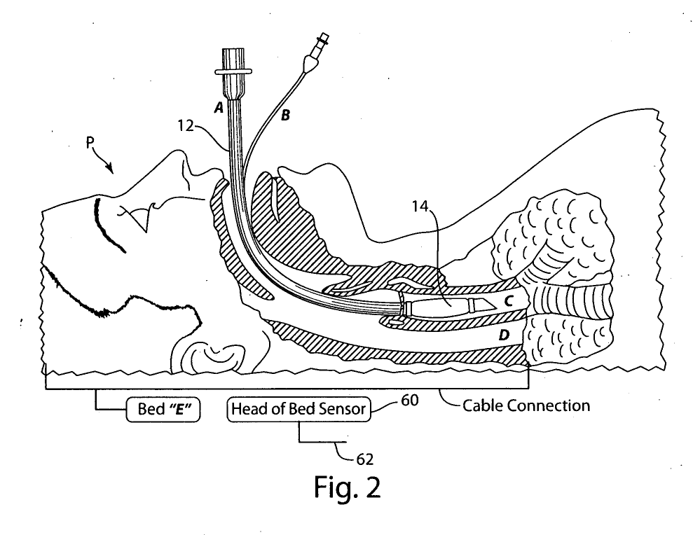

The present invention comprises a cuff pressure regulator and control system 10, partially represented in a diagram of the pneumatic circuit 15 as represented in FIG. 1. The cuff pressure regulator and control system 10 includes and is arranged to monitor and control an endotracheal tube (ETT), also called a tracheostomy tube (TT) 12 for regulating the breathing of a patient “P”, as represented in FIG. 2. The regulator and control system 10 fluidly communicates with and inflates and adjusts the pressure within a tube cuff 14 at the distal end of the ETT or TT tube 12, once the tube 12 has been inserted into the patient's trachea “C” by a medical personnel such as a respiratory technician. The technician sets a desired pressure on the regulatory and control system 10 typically within a plus or minus 2 cm of water pressure.

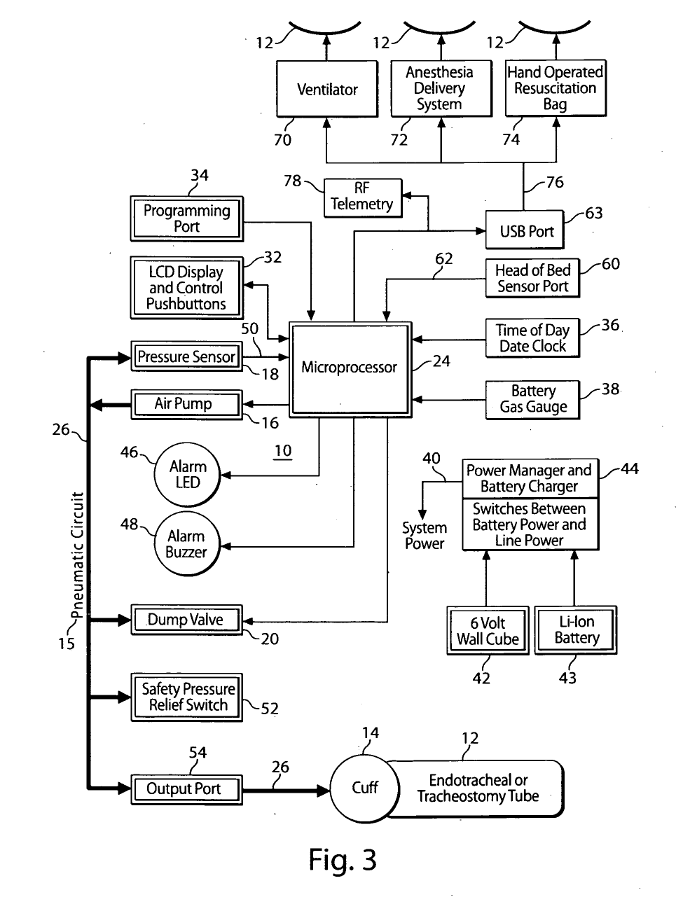

The regulator and control system 10 controls the pressure in the cuff 14 by electronic signals and through fluid connective means with a small air pump 16, a pressure sensor 18 and a dump valve 20, as represented in FIG. 3. A pressure feedback loop 50 with a programmed microprocessor controller 24 contained within the regulator and control system 10 has direct electronic signal control over both the air pump 16 and the dump valve 20.

The pressure regulator and control system 10 monitors the fluid pressure in the cuff 14 and adjusts the cuff pressure automatically to the pressure preselected by the technician. The diagram of the pneumatic circuit 15 shown in FIG. 1 shows a representation of the endotracheal tube 12 with a cuff 14 disposed annularly at one end thereof. A conduit 26 extends from the cuff 14 through the outlet port 54 and a connective line in communication with a pressure relief switch 52, and in further communication downstream with a dump valve 20, and also further downstream with a check valve 30, all in controlled, responsive fluid communication with the air pump 16. The pneumatic circuit 15 shown in FIG. 1 also displays the safety pressure relief switch 52 in communication with the dump valve 20, all in fluid communication with the cuff 14. The air pump 16 is itself electrically controlled by the microprocessor controller 24.

The pressure regulator and control system 10 is shown more specifically in FIG. 3 in a block diagram format with the central microprocessor 24 displayed centrally therein. The microprocessor 24 preferably has a liquid crystal display panel with associated proper control switches 32. The microprocessor 24 is supported by the programming port 34, a time and date clock mechanism 36 and a battery gas gauge 38. The battery gas gauge 38 is supported by a power system 40 which includes a 6 volt wall cube power supply 42, a Li-ion battery pack 43, and a power manager charger 44, having switch means for switching between battery power from the battery pack 43 and line power from the wall cube 42.

The microprocessor 24 is in electrical communication and supports an alarm light emitting diode 46 and an audible alarm buzzer 48. The microprocessor 24 controls the air pump 16. The microprocessor controlled air pump 16 is in fluid communication with the pneumatic circuit 15. The pneumatic circuit 15 is in communication with the pressure sensor 18 which reports back to the microprocessor 24 through a proper feedback circuit 50. The microprocessor 24 also controls the use of the dump valve 20. The safety pressure relief switch 52 monitors the pneumatic circuit pressure (and therefore the cuff pressure) for violation of the high limit cuff pressure trigger point. Once the cuff pressure reaches the high-limit-cuff-pressure trigger point, the safety pressure relief switch 52 energizes the dump valve 20 resulting in a lowering of the cuff pressure to safe levels without the microprocessor 24 control. The pneumatic circuit 15 will thereby be kept open to atmospheric pressure preventing dangerous cuff pressures (above 45 cm H2O) which persist for more than a few seconds. The relief is delayed to avoid deflating the cuff 14 unnecessarily when a pressure rise is only temporary, due to for example, the patient coughing or tugging of the tube 12, which would change/move the tube 14 within the patient's trachea, and thus temporarily change (increase) the tube's 14 pressure. The conduit 26 in the pneumatic circuit 15 is in direct fluid communication with the output port 54 which is in fluid communication with the tube cuff 14 disposed within the patient “P”. Such an endotracheal tube 12, is shown in aforementioned FIG. 2 wherein the cuff 14 is disposed within the patient's trachea “C”.

The microprocessor 24 of the regulator and control system 10 measures the fluid pressure in the tube cuff 14 and automatically adjusts the air pressure inside the tube cuff 14 to the level selected by the technician. That pressure level is constantly sensed by the pressure sensor 18 during the term of use of that cuff 14 within the patient's trachea “C”. The pressure in the cuff 14 is regulated by microprocessor activation of either the air pump 16 or by microprocessor activation of the dump valve 20 to lower the pressure in the tube cuff 14, all as sensed, as necessary, by the microprocessor 24 and its attached reporting units 32. The safety pressure relief switch 52 acts as a safety override to ensure that the pressure in the tube cuff 14 does not exceed 45 cm of water. The regulation and control system 10 of the present invention thus provides a constant feedback of the patient's position and cuff pressure at all times during its use.

A bed head-end angle sensor 60 is attached to the bed “E” of the patient “P”, as represented in FIG. 2, to ensure that the head of the bed is inclined at an angle of at least 30°. The sensor 60 is in communication with the microprocessor 24 through a proper circuit 62. If the angle of the head end of the patient's bed “E” is not at an angle of at least about 30 degrees, the microprocessor 24 detects the received angle-alert signal and then automatically triggers both the audible and visual alarms 48 and 46, to show and sound such improper head-of-bed angle position or condition. The audible and the visible alarm 48 and 46 are also automatically timed at periodic intervals by the microprocessor 24, as part of the intubation monitoring and control system 10, to provide a reminder to the technician to maintain the care of the patient's mouth, for swabbing while section with an antibacterial solution and/or to remove sub glottic or oral secretions from within the mouth.

A further embodiment of the intubation monitoring and control system 10 comprises a temporary initial controlled pressure surge by the air pump 16, of pressure in the ETT pr TT tube cuff from its preferred range of 20 to 40 cm of water, to a very brief, higher pressure of about 65 cm of water, so as to remove any wrinkles or creases within the ETT or TT tube cuff 14, thereby avoiding leakages past the cuff 14 in the trachea “C”.

The control unit 24 also transmits control signals to ancillary devices such as for example, a ventilator 70, an anesthesia delivery system 72 and/or a resuscitation bag 74, via a proper network 76, such signals emanating, for example, from an RF generator 78 or though a signal communicated through a USB port 63, represented in FIG. 3. The feedback through the microprocessor of 24 also automatically provides control of the ventilator 70 to thus permit changes to the ventilator output which hence readjusts its controls to the pressure and frequency of oxygen and to the delivery of medication delivery of anesthesia gases through the anesthesia delivery system 72 to the patient “P”.

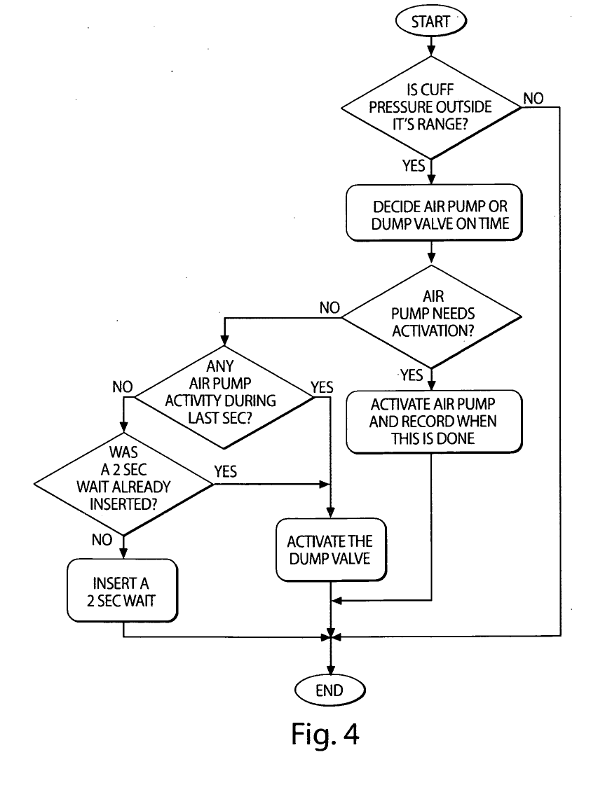

The procedure for adjusting the pressure in the cuff 14 is represented in a flow diagram in FIG. 4. The procedure is initiated where an inquiry is made by the system 10 whether the pressure of the cuff 14 is outside its required range. If the answer is no, the system investigation ceases for the moment. If the pressure of the cuff 14 is outside the required range, the system decides whether the air pump 16 should be required, or whether the dump valve 20 should be activated. If the air pump 16 needs activation, the air pump 16 is signaled by the microprocessor 24 to activate, and an electronic record is maintained when this is accomplished. The system's procedure is then ceased for that moment. If the air pump 16 does not need activation, and inquiries made whether any air pump 16 activity has taken place within the last second. If the answer to that inquiry is yes, the system 24 activates the dump valve 20 to the pressure required and the procedure ceases for that moment. If the answer to that inquiry as to whether any air pump activity occurred during the last second is no, an inquiry is made of the system 10 as to whether there was a 2 second wait already inserted within the procedure. If the answer is yes, the system 24 activates the dump valve 20 and the procedure ceases for that moment. If the answer to the question was a 2 second wait already inserted into the procedure is no, the system 10 is then instructed to wait a further 2 seconds, and the system's inquiry procedure for that moment ceases. Through a proper time interval, the inquiry is again recycled and the inquiry is made as to whether the pressure of the cuff 14 is outside of its proper range.

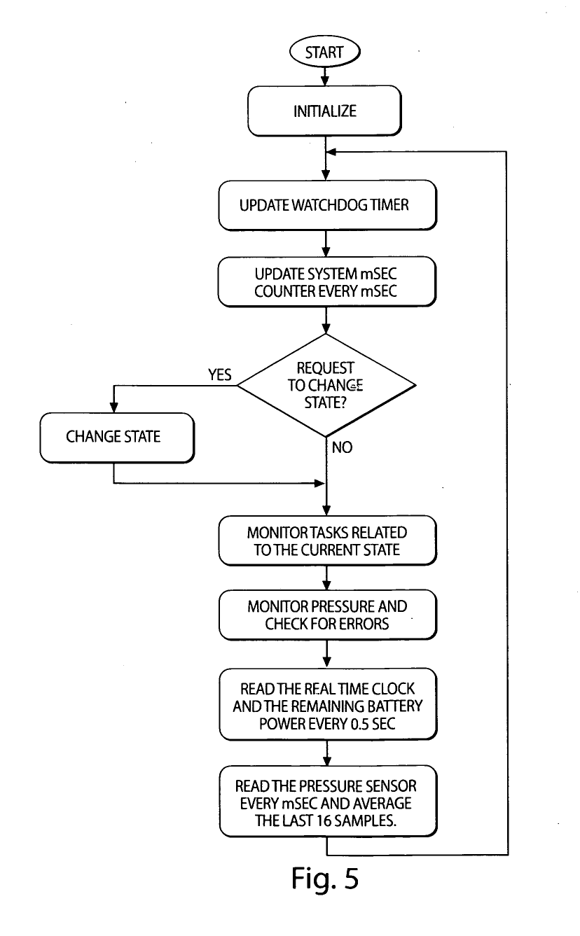

The procedure for the control unit 24 is represented in a flow diagram format shown in FIG. 5, wherein the system is initialized, and updated as to its particular time. The system updates itself every millisecond. The system inquires as to whether it is necessary to change the state of the protocol controlling the systems functions. If the answer to that question is yes, the control unit or microprocessor 24 signals the necessary change of state to occur. If the answer to whether it is necessary to change the state of the protocol controlling the systems functions is no, the system instructs the microprocessor to continue to monitor the tasks related to the current state of the system components. This involves a continuing monitoring of the pressure to check for variations and errors. A real-time clock is read and the battery power is monitored every one half second. The pressure sensor is read every millisecond and averages are calculated over the previous 16 samples taken.

In the use of the present invention, when a patient is actively thrashing about, or hiccupping, or in any way making movements that tend to squeeze on the cuff, 14 or even if the patient is still, but the ventilator 70 or anesthesia delivery instrument 72 is exerting a pressure on the cuff 14 during the patient's inspiratory phases, the result is a rise in the cuff pressure sensed by the system. If the system software were to adjust the pressure immediately by removing air from the cuff, when the interfering movements or the respiratory pressure exerted by the ventilator 70 or anesthesia delivery instrument 72 are removed, the result would likely be that the cuff pressure would wind up below the desired set point pressure. This is a dangerous outcome because too low a cuff pressure may well allow silent aspiration of sub-glottic fluids that have the potential to result in giving the patient pneumonia, sometimes referred to as the VAP, or ventilator associated pneumonia. In order to overcome this result, the system has software included to detect these interferences and to wait a short time to allow the interference to dissipate on its own. In this way air is not removed from the cuff during such brief interferences and the danger that results in too low a cuff pressure is averted. This delay is set to 2 seconds but may be programmed into the microprocessor for different delay times by a skilled operator. Some embodiments of cuff regulators in the prior art have attempted to overcome the cuff pressure variations that occur when patients are active, or even during the normal inspiratory cycles by a design of a fast servo-regulated control loop. However, because of the unpredictability of these pressure altering events, the inevitable result of such attempts is that because pressure is reduced even for a very short period of time of about 0.3 seconds or so, which will allow either singularly or in conjunction with multiple occurrences of these short periods, the silent aspiration of sub-glottic fluids that can infect the patient's lungs causing pneumonia. The present invention overcomes these negative results by delaying cuff pressure adjustments in deference to the ventilator and patient involvement in the rise and fall of monitored cuff pressure.

Because air pumps themselves have variations in response characteristics, the pneumatic circuit formed by the cuff 14 situated within the patient's trachea and the system of the present invention is able to present variations in its compliance from patient to patient. Also, because of the environmental effects of temperature and altitude, the pneumatic circuit characteristics are likely variable upon each use. These variations in response characteristics and compliance can adversely affect the ability of the system to adjust the cuff pressure properly. For instance, a strong pump may have enough air to the cuff to increase the cuff pressure by about 2 cm of water during one activation cycle. Whereas a weak pump may require several such pump activations to raise the cuff pressure an equal amount. The problem presented to the control mechanism by these variations in response characteristics and compliance is that the control mechanism cannot rely on a fixed relationship between a single activation and a reliable cuff pressure change. The present invention includes an adaptive algorithm within the microprocessor 24 whereby the relationship between the pump activations and the resulting cuff pressure changes is detected and learned by the microprocessor 24 and then, the time duration of single pump activations and the frequency of such pump activations are adjusted by the software in the system's microprocessor 24, to accommodate variations in these characteristics.
a. The software uses a base “Air Pump On” Time as the minimum On Time for the Air Pump. The software then can increase this base On Time in incremental manner towards a maximum On Time if warranted by the resulting cuff pressure change. Two variables are used for this algorithm:
   i. T: The previous Air Pump On Time
   i. N: The number of times the Air Pump was activated per each bracket. [Brackets are fixed in the software to define the magnitude of error between the present cuff pressure and the desired set point cuff pressure. For example, a bracket may be created to be for those errors between 1 and 2 cmH2O from the set point. Whereas a second bracket may be created to be for those errors between 4 and 2 cmH2O from the desired set point pressure, and so on.]
   ii. T is set to 0 to indicate no previous Air Pump On Time available if one of the following conditions is met:
      i. Power up
      ii. Entering a new mode (state)
      iii. Enabling or disabling the Cuff pressure adjustment
      iv. New Cuff set pressure
      v. Cuff pressure within the tolerance (no Air Pump or Dump Valve)
      vi. The Dump Valve needs activation
      vii. Cuff pressure discrepancy is greater than 126 A to D counts
   iii. T is set to a base “Air Pump On” Time and the counter N is reset to 0 when one of the following conditions is met:
      i. Going to the next bracket
      ii. Going to the previous bracket
   iv. The “Air Pump On” Time equals T+(N*U). Integer N is incremented by one each time the pump is activated while the cuff pressure error remains within a single bracket. Integer N can be incremented up to a maximum of 20. Variable U is either 2 when cuff pressure error is 33 A to D counts (an error of about 4 cmH20) or less or 3 when cuff pressure error is greater than 33 A to D counts.
   v. The final “Air Pump On” Time used is subject to a maximum value. This maximum “Air Pump On” Time is either 45 mSec when the cuff pressure error is 59 A to D counts (about 7 cmH2O error) or less or 65 mSec when the cuff pressure error is greater than 59 A to D counts.
      Should a failure within the system 10 occur that causes the air pump 16 to run continuously, a dangerously high cuff pressure greater than 100 cmH2O can be achieved within a couple of seconds. If this condition goes undetected and corrected in as little as 15 minutes or so, this high a cuff pressure can cause the beginning of trauma in the patient's trachea and/or voice box. Left unchecked for long durations high cuff pressure has been found to cause ulcerations and fistulas within the patient's trachea.
vi. Simply using a pressure relief valve as most people “skilled” in the art may do has the effect of “regulating” the cuff pressure at that elevated pressure by its on/off nature. The FDA did not like that because that pressure level may do harm to the patient. A novel approach to address the FDA concerns was adopted and is presented as a part of this invention. The trigger point is still a high cuff pressure, but it only is high for a few seconds. Once triggered, the pressure switch 52 contacts cause the dump valve 20 to open venting the pneumatic circuit 15 to atmosphere. Thereby lowering the cuff pressure. The relief valve approach does not lower the cuff pressure, it simply limits the cuff pressure.
vii. The safety overpressure system described in “a” above detects and mitigates without the need for properly running software. It forms a safety control beyond the manipulation of software forming a patient safeguard.
viii. The safety overpressure system includes the detection ability of a settable pressure switch that once triggered activates the system's pneumatic valve resulting in opening the pneumatic circuit 15 to atmosphere.
ix. The design relies on the natural delay or hysteresis in the pressure switch 52 to keep the pneumatic valve 20 open for an extended period. This extended open period results in the lowering of the cuff pressure much below the trigger point. This is how the resulting cuff pressure can be lowered to levels of 45 cmH2O or so even though the opening pressure point of the safety pressure switch may be set to as high as 100 cmH2O or more.

The bundling of strategies to improve intubated patient outcomes is well reported in the literature. Care bundles vary in the items that are included but generally the bundles include among several other items these next three interventions as recommended, to improve intubated patient outcomes: (1) Cuff Pressure Management; (2) Head of Bed Monitoring (HOB): An accessory device connects to the PYTON via cable, and straps to the head rails of the patient's bed using a Velcro strap or other means. The device electronically measures (two magnetic Hall Effect sensors orthogonally positioned) the angle made between the bed head rails and Earth's gravity. The system 10 runs the alarms if the HOB is less than 30 degrees for a fixed time; and (3) Mouth Care and Sub-Glottic Suction: The present invention provides for a user selectable timer that alerts the care staff to the need for mouth care and/or sub-glottic or tracheal suctioning. The present invention thus includes in a single device automatic control or monitoring for all three of the “bundled” items listed above.

We claim:

1. An endotracheal tube or tracheostomy tube monitoring and control system for the safe and effective use of a pressurizable annular cuff about the distal end of that tube arranged within the larynx or trachea of a human patient comprising:
a control unit for the monitoring and control of the tube cuff in communication therewith;
a pressure sensor in communication with the control unit and with an empowered air pump and associated a dump valve; and
a feedback circuit connected to an ancillary device supporting the patient, so as to control time and pressure responsive use of the ancillary device relative to pressure changes in the tracheal cuff in the patient.

2. The endotracheal tube or tracheostomy tube monitoring and control system as recited in claim 1, wherein the ancillary devices are selected from the group consisting of: a ventilator, an anesthesia machine and a resuscitation bag.

3. The endotracheal tube or tracheostomy tube monitoring and control system as recited in claim 1, wherein the pressure in the cuff is pre-set and maintained at a selected value by an system operator, and wherein the system provides feedback of pressure within the cuff and acts to dump pressure from that cuff after a predetermined delay to avoid unnecessary and potentially damaging pressure increase.

4. The endotracheal tube or tracheostomy tube monitoring and control system as recited in claim 1, including:
a self-test control unit protocol which automatically checks operation of the pneumatic system control components to assure that the microprocessor continues to have control over system integrity for feedback to the ancillary device.

5. The endotracheal tube or tracheostomy tube monitoring and control system as recited in claim 1, including:
a pressure sensor in fluid communication with a dump valve and an air pump, controlled by a control unit arranged to delay pressure changes in the ancillary device connected to the system and to delay pressure changes effected by the system on the cuff.

6. The endotracheal tube or tracheostomy tube monitoring and control system as recited in claim 1, including:
a programming port for inputting to the control unit, instructions for the pressure and frequency overrides on the system's ancillary device.

7. The endotracheal tube or tracheostomy tube monitoring and control system as recited in claim 1, including:
a dual alarm arrangement for establishing a periodic review of a patient utilizing the system.

8. The endotracheal tube or tracheostomy tube monitoring and control system as recited in claim 7, including:
a pneumatic circuit controlled by the control unit, the circuit consisting of a pressure sensor, an air pump, a dump valve, a safety pressure relief switch attached to the dump valve, and an output port, all in controlled fluid communication with the cuff.

9. The endotracheal tube or tracheostomy tube monitoring and control system as recited in claim 8, wherein the output port communicates directly with the cuff, the output port being pressurized directly by the air pump which is controlled by the pressure sensor and the control unit.

10. The endotracheal tube or tracheostomy tube monitoring and control system as recited in claim 9, including:
a safety pressure relief switch to override the microprocessor control should cuff pressures reach dangerous levels above 45 cm H2O.

11. A method to control ancillary devices supporting a patient having an endotracheal tube inserted within the patient's trachea, the method comprising:
a system controlled by a control unit, which unit controls timed pressure intervention in a cuff on the tracheal tube;
a delay arrangement within the control unit to delay an decrease in pressure in the cuff in response to a signal received by the control unit, so as to avoid pressure changes responsive to temporary increases in cuff pressure to prevent pressure change operation of ancillarily connected devices.

12. The method as recited in claim 10, wherein the delay of a decrease in pressure in the cuff in response to a received signal by the control unit of an improper increase in pressure in the cuff is about 2 seconds.

13. The method as recited in claim 10, wherein the delay of a decrease in pressure in the cuff in response to a received signal by the control unit of an improper increase in pressure in the cuff is about 5 seconds to about 10 seconds.

14. A method for monitoring and controlling use of an endotracheal or tracheal tube cuff in a patient's trachea of a patient lying in a bed, comprising:
attaching a pressurized line to a tube cuff for engaging the patient's trachea;
monitoring and controlling the pressure in the cuff in the patient by a monitor and control system;
limiting the pressure in the cuff to about 45 inches of water;
sounding a patient monitoring alarm by the system, to remind a patient assist personnel to check and cleanse the patient's airway;
sounding a patient monitoring alarm by the system, if the angle of the bed is less than about 30 degrees from horizontal;
overriding control from the microprocessor by the safety pressure relief switch in response to a received danger signal, should cuff pressures rise to dangerous levels; and
controlling ancillary devices from introducing pressure increases into the cuff.

15. The method for monitoring and controlling use of an endotracheal or tracheal tube cuff in a patient's trachea as recited in claim 14, including:
communication with an ancillary patient device to provide an immediate indication of patient discomfort.

16. The method for monitoring and controlling use of an endotracheal or tracheal tube cuff in a patient's trachea as recited in claim 14, wherein the ancillary device is a ventilator.

17. The method for monitoring and controlling use of an endotracheal or tracheal tube cuff in a patient's trachea as recited in claim 14, wherein the ancillary device is a anesthesia device.

18. The method for monitoring and controlling use of an endotracheal or tracheal tube cuff in a patient's trachea as recited in claim 14, including:
lowering the cuff pressure after a timed delay after the received danger signal has been picked up by the microprocessor.

19. The method for monitoring and controlling use of an endotracheal or tracheal tube cuff in a patient's trachea as recited in claim 15, wherein the time delay is about 2 seconds before pressure is reduced.

20. The method for monitoring and controlling use of an endotracheal or tracheal tube cuff in a patient's trachea as recited in claim 15, wherein the time delay is about 5 to 10 seconds before pressure is reduced.
